(19) United States
(12) Patent Application Publication
(10) Pub. No.: US 2010/0006108 A1
(43) Pub. Date: Jan. 14, 2010

(54) ANTI-SNORE GARMENT

(76) Inventor: Sean Patrick Kerklaan, Vancouver (CA)

Correspondence Address: NEXUS LAW GROUP LLP, 1500 - 701 WEST GEORGIA STREET, VANCOUVER, BC, V7Y 1C6, CA

(21) Appl. No.: 12/172,964

(22) Filed: Jul. 14, 2008

(51) Int. Cl. A61F 5/56

(52) U.S. Cl. 128/848

(57) ABSTRACT

An anti-snore garment in the form of a shirt having at least three cylindrical bumpers removably fitted within bumper pockets on the back of the shirt. The tops of the bumper pockets are horizontally aligned with a wearer's shoulder blades. The bumpers are adapted to resist rotation of the wearer into a supine position.

### FIELD OF THE INVENTION

The present invention relates to the field of devices for limiting a user's range of motion while sleeping; and more particularly a device for aiding in limiting snoring and positional sleep apnea by preventing a user from sleeping on their back.

### BACKGROUND OF THE INVENTION

In these busy times, it is more and more difficult for people to get a sufficient amount of sleep. In addition to having to sacrifice sleep in order to fit everything into a day, many people simply suffer from poor quality sleep. Snoring is one of the most common causes of a poor quality sleep, both for the person doing the snoring and for those in proximity to them.

Snoring is a sound emitted by a person while sleeping and is caused by vibration of a person's respiratory structure during breathing due to obstructed air movement. Snoring is usually caused by one or more factors; for example, throat weakness which results in the throat closing during sleep, a mispositioned jaw, fat gathering in and around the throat, or obstruction in the nasal passageway. The actual noise emitted by the snorer can vary substantially, from a soft rasp to a loud unpleasant rumble. Regardless of the noise level or intensity, snoring is a problem and can be a symptom of the more significant problem known as Obstructive Sleep Apnea (“OSA”).

Sleep apnea is a sleep disorder characterized by pauses in breathing during sleep. OSA is the most common category of sleep-disordered breathing, the widespread symptom of which is loud snoring, and also includes restless sleep and drowsiness during the day. OSA can vary from mild, occasional sleep apnea, to chronic, severe obstructive sleep apnea that requires treatment to prevent sleep deprivation and other complications including the possibility of congestive heart failure. Positional Sleep Apnea is sleep apnea caused by the position adopted by a person while sleeping (sleeping on their back).

Snoring and sleep apnea cause problems for both the individual suffering from the affliction and for any others sleeping in close proximity with them. The typical pattern is for the person snoring to wake any others in proximity and for the person woken by the snorer to then wake the snorer in order to get them to roll on their side with the hope that the snoring will stop. This results in disrupted sleep for all involved. This poor quality sleep can impact a person's health and can have a significant effect on their ability to function during the day as they are more prone to drowsiness, irritability, and lack of focus. It can lead to decreased libido and can affect marital relations. Clearly, snoring is a problem, one that people have been trying to solve for hundreds of years.

There are numerous treatments for snoring. At a basic level they can involve lifestyle changes, such as avoiding alcohol or muscle relaxants, losing weight, and quitting smoking. These lifestyle changes may or may not have any relevance to a given snorer. More complicated and costly solutions have also been developed, including specially made dental appliances for controlling the positioning of the wearer's jaw and tongue, specially designed machines that pump a controlled stream of air through a flexible hose to a mask worn by the user, or even surgery. These solutions suffer from a variety of problems including their high cost, the inconvenience to the user and the risks associated with them, in particular the surgery, which can cause the problem to worsen.

The easiest and most commonly used solution to snoring and positional sleep apnea is for the individual to sleep on their side. Sleeping on your side helps prevent snoring and positional sleep apnea as the airway collapsing effect of gravity is not as strong when a person is on their side. The difficulty is ensuring that the person remains on their side throughout the night.

Numerous devices have been developed in order to keep a person positioned on their side. For example, U.S. Pat. No. 132,500 issued to Sullivan in 1872 for an “Apparatus for Preventing a Person While Asleep From Turning on His Back”. The device taught by Sullivan comprises an arch sprung from two shoulder pads which is fitted to a harness to be worn by a user. The arch is positioned in the center of a user's back and is formed of wire covered with cloth. This is a cumbersome device with a complicated series of straps for retaining the arch in the proper position. Ultimately, given the positioning of the arch, it will not function to keep a person on their side, but rather prevents them from lying flat on their back mainly through discomfort.

There are many designs that rely on the principal of discomfort. U.S. Pat. No. 663,825 teaches a “Shoulder Brace and Anti-snoring Attachment”. The shoulder harness functions both to assist with a wearer's posture and to hold the “Anti-snoring Attachment” in place. The attachment is a metal prod, having several projections, preferably four, with balls or knobs on their ends to prevent injury. When a person wearing the device rolls onto their back, the projections will cause discomfort thereby causing the person to change their position so as to alleviate the pressure. U.S. Pat. No. 898,379 teaches an “Antisnoring Device” comprising a harness and a compressible ball, the ball being held in position in the middle of the wearer's back. It is also designed to be uncomfortable so as to cause the wearer to wake up and move back onto their side. This idea was further refined in U.S. Pat. No. 2,304,235 which teaches a sleeping garment having a pocket to hold the ball in position. This overcomes problems associated with the harness, but still relies on discomfort to keep a person off their back. U.S. Pat. No. 5,036,865 teaches a similar sleeping garment, however, this garment has two balls or sensors in pockets, one positioned proximate each shoulder blade. While a simple tennis ball can be placed in the pockets so as to cause discomfort should the wearer roll onto their back, the '865 patent teaches the use of a sensor coupled with an audible or vibrating alarm. When the wearer rolls so as to cause the sensor to be compressed, the sensor emits an alarm waking the person in order to get them to roll back on their side. The device taught in U.S. Pat. No. 5,383,475 also relies on discomfort; however it comes in the form of a belt for wearing about a person's chest. The belt has a plurality of upstanding semi-rigid stems located in wells on the belt. The stems have a height slightly lower than the depth of the well so that in an inactivated position they do not cause the wearer any discomfort. Should the wearer roll onto their back the pressure will force the stems into the wearers back, causing discomfort and waking the wearer and prompting them to move back on their side.

The main deficiency with all these devices is their reliance on the principal of discomfort in order to cause the wearer to move back onto their side. Once the wearer reaches a supine position they will eventually wake from the discomfort and roll back onto their side. This results in disruption to the users sleep and potentially to that of anyone sleeping in their vicinity as depending on the wearers sensitivity level (and the effectiveness of the various devices) they may remain on their back long enough to start snoring.

U.S. Pat. No. 6,357,444 teaches a motion limiting device which attempts to overcome this problem with the prior art. The '444 patent teaches a large pad connected by way of a harness to a person, the pad being sufficiently sized to prevent a person from rolling into the supine position. While moving away from the principal of discomfort to wake a person and cause them to resume an appropriate sleeping position, the size and connection method of the pad is cumbersome and inconvenient. The pad is not designed around comfort; it is a large, awkward back pad secured with uncomfortable straps.

It is therefore an object of an embodiment of the present invention to provide an apparatus for preventing a person from rolling on their back that is less costly, complicated, cumbersome and uncomfortable than those taught in the prior art.

Other objects of embodiments of the invention will be apparent from the description that follows.

### SUMMARY OF THE INVENTION

The anti-snore device of the present invention is designed to provide a person with a comfortable solution for combating snoring and sleep apnea. It is designed to prevent a wearer from rolling from a lateral position to a supine position thereby keeping the wearer from adopting the more snore and positional sleep apnea prone supine position and doing so without disrupting the wearer's sleep.

According to the present invention there is provided a sleeping garment having at least three pockets positioned across the back at a position corresponding to just below the wearer's shoulder blades. A bumper, preferably in the form of a cylindrical pad, is positioned in each of the pockets.

In another aspect, each bumper has a diameter of 3 to 4 inches and a length of 3.5 inches. The bumpers are removable from a resealable opening in each bumper pocket.

Other aspects of the invention will be appreciated by reference to the detailed description of the preferred embodiment and to the claims that follow.

### BRIEF DESCRIPTION OF THE DRAWINGS

These and other features of the invention will become more apparent from the following description in which reference is made to the appended drawings and wherein:

FIG. 1 is a rear view of a person wearing the anti-snore garment of the present invention;

FIG. 2 is a top view of a person wearing the anti-snore garment of the present invention lying on their side;

FIG. 3 is a top view of a person wearing the anti-snore garment of the present invention lying on their side, showing the garment preventing the person from rolling onto their back;

### DETAILED DESCRIPTION OF THE PREFERRED EMBODIMENT

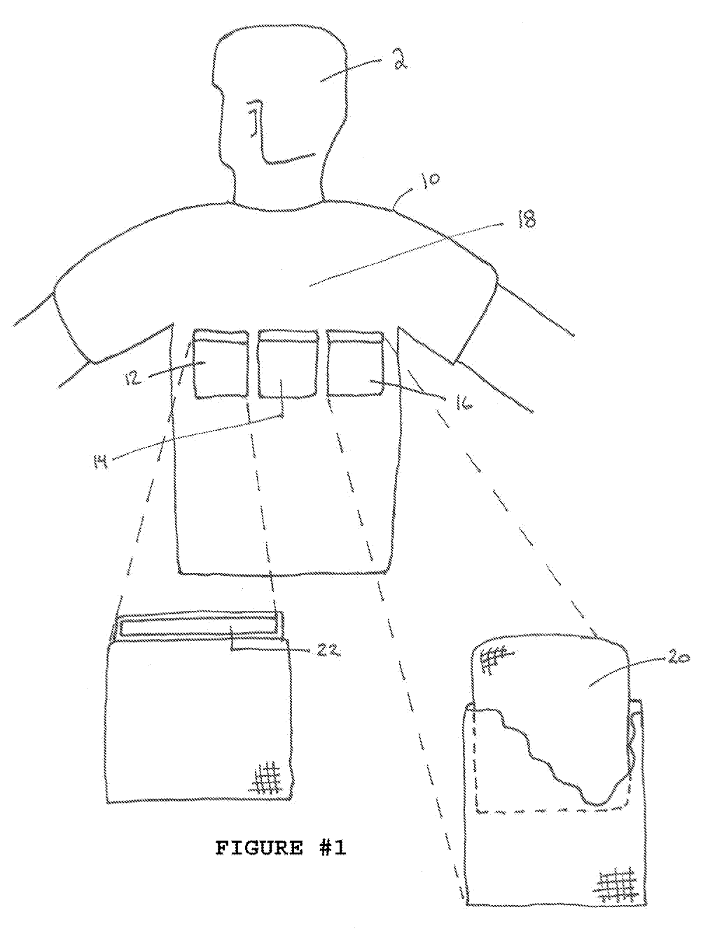

The preferred embodiment of an anti-snore garment 10 is shown being worn by a wearer 2 in FIG. 1. The anti-snore garment 10, in the form of a sleep shirt, which could have short, long, or no sleeves, has at least three bumper pockets 12, 14, 16 independently attached to the back 18 of the garment 10. Preferably, the bumper pockets 12 are attached to the garment in a position so that when the garment is worn, the bumper pockets 12, 14, 16 are each vertically connected so that the top of the pockets are horizontally aligned with the bottom of the wearer's shoulder blades. The pockets are approximately laterally centered on the back of the garment as shown in FIG. 1

A bumper 20 in the form of a cylindrical cushion is placed in each bumper pocket 12, 14, 16. The bumper pockets are attached to the garment at the bottoms and sides, the top of each bumper pocket being selectively sealable by way of Velcro® or other suitable fastener. It is also contemplated that the opening of the pocket could be located at the bottom rather than the top. Having a resealable opening allows the bumpers 20 to be easily removed for cleaning of the garment or for wearing it at times when the bumpers are not needed. When a bumper 20 is inserted into its respective pocket, it sits just below the level of the shoulder blade of the wearer. This is the preferred position to support the wearer's body from rolling into a supine position while keeping the garment 10 comfortable to sleep in.

Each bumper 20 is allowed free lateral movement in its respective bumper pocket 12, 14, 16 to the extent allowed by the confines of the pocket itself. Preferably, the pockets are sized so as to be small enough that the bumpers are not able to move around too much and become misaligned, while at the same time offering enough room for the bumpers to move while the wearer sleeps in a lateral position to ensure the wearer does not feel prodded by the bumpers while in a healthy lateral sleeping position. While it is contemplated that other bumper shapes could be used, the cylindrical form works best as it allows rotation of the bumper to better translate the resistance from the lower bumper up to the top bumper and from the top bumper to the top shoulder where it will be more effective in preventing the rolling motion

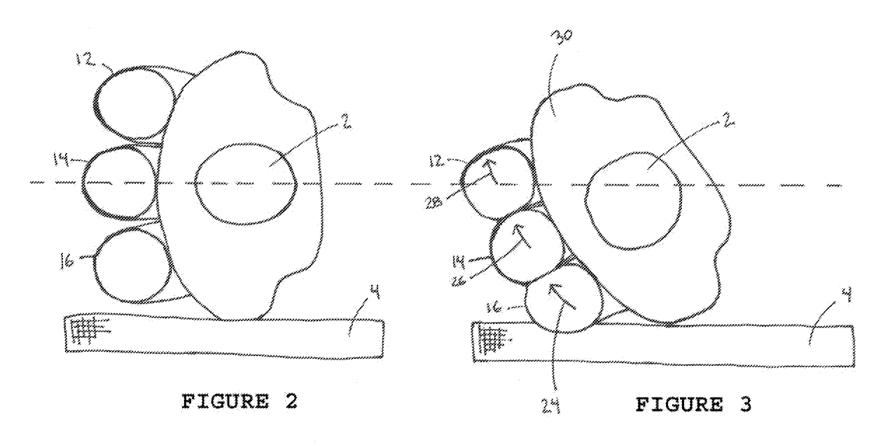

Use of the anti-snore garment will now be described with reference to FIGS. 2 and 3. In FIG. 2, the wearer 2 is shown lying on their side on a bed 4. The bumpers 20 in their bumper pockets 12, 14, 16 are able to hang loosely causing no discomfort to the wearer 2. Should the wearer start to roll onto their back, as illustrated in FIG. 3, the bumpers begin to press against each other causing a resistance to any further turning of the wearer to a supine position. The bumper in bumper pocket 16 is first to come into contact with the bed 4 causing it to rotate laterally in the direction shown by arrow 24 and come into contact with the bumper in bumper pocket 14. The bumper in bumper pocket 14 is in turn forced to rotate in direction 26 into the bumper in bumper pocket 12, which is forced to rotate in direction 28. In addition to applying pressure against the adjacent bumper, each bumper applies pressure against the wearer's back. The bumper opposite the point of contact with the bed 4, which in this case is bumper 12 located proximate the wearer's left side exerts a rotational pressure against the wearer's top side 30 counter to the wearer's direction of rotation into the supine position. It is this transfer of resistance from the lowest bumper to the top bumper which makes the present invention so effective.

While the invention has been described with three pockets and bumpers, 4 or more pockets and bumpers are necessary for larger individuals in order to ensure adequate support while maintaining comfort. Preferably the bumpers are made of a dense PE Styrofoam that does not compress to any great extent and which does not absorb sweat, but yet is light and durable.

Preferably, the bumpers measure 3.5 inches in length and 3 to 4 inches in diameter. The smaller diameter is sufficient for individuals wearing a small or medium sized garment, while 4 inch diameter bumpers are best for larger individuals (wearing large or extra large sized garments). The sides of adjacent bumper pockets are preferably spaced 0.5 inches apart from one another. This spacing has been found to provide the best fit while quickly offering sufficient support as the wearer shifts towards a supine position

The garment is designed to maximize comfort and effectiveness so that a person wearing the garment is able to sleep through the night and not be woken up by an uncomfortable jab in the back. The bumpers gently resist a wearer's rotational movement into a supine position thereby keeping them in the preferred lateral position in order to minimize snoring and positional sleep apnea. Wearing the sleep garment of the invention is better than getting elbowed from a partner and better than the previous inventions, the bumpers gently resist the roll and allow the wearer to enjoy a great night sleep.

It will be appreciated by those skilled in the art that the preferred and alternative embodiments have been described in some detail but that certain modifications may be practiced without departing from the principles of the invention.

What is claimed is:

1. An anti-snore garment for preventing a wearer from rolling into a supine position comprising:
a shirt having at least three pockets fixedly connected to the back of the garment, each of said pockets having a bumper removably fitted therein.

2. The anti-snore garment of claim 1 wherein said bumpers being cylindrically shaped.

3. The anti-snore garment of claim 2 wherein said bumpers being vertically aligned such that the tops of each bumper are horizontally aligned with the bottom of a wearer's shoulder blades.

4. The anti-snore garment of claim 2 wherein said bumpers having a diameter of at least 3 inches.

5. The anti-snore garment of claim 1 wherein said at least three pockets having three sides connected to said back of said garment and a fourth side releasably connectable to the back of said garment so as to enclose said bumpers.
